(19) United States
(12) Patent Application Publication
(10) Pub. No.: US 2023/0285804 A1
(43) Pub. Date: Sep. 14, 2023

(54) ACTION FEEDBACK METHOD AND DEVICE

(71) Applicant: CHENGDU FIT-FUTURE TECHNOLOGY CO., LTD., Chengdu (CN)

(72) Inventors: Weitao XI, Chengdu (CN); Bowen ZHANG, Chengdu (CN); Zhiling ZHONG, Chengdu (CN); Yong FU, Chengdu (CN)

(73) Assignee: CHENGDU FIT-FUTURE TECHNOLOGY CO., LTD., Chengdu (CN)

(21) Appl. No.: 18/045,462

(22) Filed: Oct. 10, 2022

(30) Foreign Application Priority Data
Mar. 14, 2022 (CN) 202210249613.3

(51) Int. Cl. A63B 24/00

(52) U.S. Cl. CPC A63B 24/0006; A63B 24/0062; A63B 2024/0012; A63B 2024/0068

(57) ABSTRACT

An action feedback method and device are provided. The method includes: judging whether an action is safe; and obtaining motion parameter data corresponding to the action in response to a judgment that the action is safe, and conducting parameter feedback according to the motion parameter data.

### CROSS-REFERENCE TO RELATED APPLICATIONS

The application claims priority to Chinese Patent Application No. 202210249613.3, filed on Mar. 14, 2022, the contents of which are hereby incorporated by reference to its entirety.

### TECHNICAL FIELD

The present disclosure generally relates to fitness field, and in particular to an action feedback method and device.

### BACKGROUND

With the improvement of living standard, keeping healthy has become a basic pursuit of people. However, in real life, people are at a loss about the types of fitness exercises they should do and how to choose a right fitness program to achieve their goals. Therefore, an intelligent fitness system that can make fitness plans according to users' own conditions has a broad market prospect at present. However, it is important for the intelligent fitness system to detect error operation, error action, and/or nonstandard action when users exercise according to the fitness plans. Particularly, error operation may lead to unsafe fitness process, the action error may lead to a fitness failure and may also lead to dangerous accidents, nonstandard actions may not achieve good fitness effect. At the same time, when users do a group of actions, the performance of each action in the group may be relatively poor, the users may not adapt to the specified action intensity, which may reduce senses of use experience of the users and make it inconvenient for long-term use.

Therefore, an action feedback method and device are proposed, which is helpful to cover more dimensions of users' actions and improve the accuracy of action feedback.

### SUMMARY

One aspect of the embodiments of the present disclosure aims to provide an action feedback method, wherein the method includes: judging whether an action is safe; obtaining motion parameter data corresponding to the action in response to a judgment that the action is safe, and conducting parameter feedback according to the motion parameter data.

In some embodiments, an action attribute corresponding to the action includes centripetal training or centrifugal training, the motion parameter data includes motion distance data, and the parameter feedback includes motion distance feedback; and the obtaining motion parameter data corresponding to the action and conducting parameter feedback according to the motion parameter data includes obtaining the motion distance data, and conducting the motion distance feedback according to the motion distance data.

In some embodiments, when the action attribute corresponding to the action is the centripetal training, the obtaining the motion distance data, and conducting the motion distance feedback according to the motion distance data includes: obtaining a centripetal motion start point k and a centripetal motion end point k corresponding to the action; obtaining a centripetal motion distance k according to the centripetal motion start point k and the centripetal motion end point k; obtaining a current cumulative centripetal motion distance according to the centripetal motion distance k; and comparing the current cumulative centripetal motion distance with a preset centripetal motion distance to judge the action error or standard or non-standard; when the action attribute corresponding to the action is the centrifugal training, the obtaining the motion distance data and conducting the motion distance feedback according to the motion distance data includes: obtaining a centrifugal motion start point k and a centrifugal motion end point k corresponding to the action; obtaining a centrifugal motion distance k according to the centrifugal motion start point k and the centrifugal motion end point k; obtaining a current cumulative centrifugal motion distance according to the centrifugal motion distance k; and comparing the current cumulative centrifugal motion distance with a preset centrifugal motion distance to judge whether the action is error, or standard or non-standard.

In some embodiments, the action attribute corresponding to the action includes centripetal training or centrifugal training, the motion parameter data includes motion time data, and the parameter feedback includes motion time feedback; and the obtaining motion parameter data corresponding to the action and conducting parameter feedback according to the motion parameter data includes obtaining the motion time data and conducting the motion time feedback according to the motion time data.

In some embodiments, when the action attribute corresponding to the action is the centripetal training, the obtaining the motion time data and conducting the motion time feedback according to the motion time data includes: obtaining a centripetal motion start point k and a centripetal motion end point k corresponding to the action;
obtaining a centripetal motion time k according to the centripetal motion start point k and the centripetal motion end point k; and
prompting to speed up the action or slow down the action by comparing the centripetal motion time k with a preset centripetal motion time; and
when the action attribute corresponding to the action is the centrifugal training, the obtaining the motion time data and conducting the motion time feedback according to the motion time data includes: obtaining a centrifugal motion start point k and a centrifugal motion end point k corresponding to the action; obtaining a centrifugal motion time k according to the centrifugal motion start point k and the centrifugal motion end point k; and prompting to speed up the action or slow down the action by comparing the centrifugal motion time k with a preset centrifugal motion time.

In some embodiments, when the centripetal motion includes at least two centripetal motion end points and the centrifugal motion includes at least two centrifugal motion start points, and the obtaining the motion time data and conducting the motion time feedback according to the motion time data includes: obtaining an equal length motion time according to a time difference between a last centripetal motion end point corresponding to the centripetal motion and a first centrifugal motion start point corresponding to the centrifugal motion; and comparing the equal length motion time with a preset equal length time, and prompting that a next motion state corresponding to the action is retracted or maintained.

In some embodiments, when the centripetal motion includes at least two centripetal motion distances and the centrifugal motion includes at least two centrifugal motion distances, the obtaining the motion distance data, and conducting the motion distance feedback according to the motion distance data includes: obtaining comprehensive feedback according to motion distance feedback of a last centripetal motion distance and a first centrifugal motion distance.

In some embodiments, the motion parameter data includes motion speed data; and obtaining the motion speed data according to the motion distance data and the motion time data corresponding to the motion distance data, and conducting motion weight feedback according to the motion speed data.

In some embodiments, the conducting the motion weight feedback according to the motion speed data includes: in a group of actions, obtaining an action satisfying preset conditions, the preset conditions including: the comprehensive feedback corresponding to the action is that the action is standard or non-standard, and the action attribute corresponding to the action is the same as a preset action attribute; obtaining an average motion speed of the action satisfying the preset conditions; and comparing the average motion speed with a preset average speed to determine whether to adjust a motion weight corresponding to the action.

In some embodiments, the preset average speed includes a preset average centripetal speed and a preset average centrifugal speed; when the action attribute corresponding to the action is the centripetal training, if the average motion speed corresponding to the centripetal training is greater than a maximum value of the preset average centripetal speed, increasing the motion weight; if the average motion speed corresponding to the centripetal training is less than a minimum value of the preset average centripetal speed, reducing the motion weight; when the action attribute corresponding to the action is the centrifugal training, if the average motion speed corresponding to the centrifugal training is greater than a maximum value of the preset average centrifugal speed, reducing the motion weight; if the average motion speed corresponding to the centrifugal training is less than a minimum value of the preset average centrifugal speed, increasing the motion weight.

One aspect of the present disclosure provides an action feedback device, including at least one storage medium and at least one processor: at least one storage device including a group of instructions; at least one processor communicating with the at least one storage device, wherein when executing the group of instructions, the at least one processor is configured to enable the system to perform operations, including: judging whether an action is safe; obtaining motion parameter data corresponding to the action in response to a judgment that the action is safe, and conducting parameter feedback according to the motion parameter data.

In some embodiments, an action attribute corresponding to the action includes centripetal training or centrifugal training, the motion parameter data includes motion distance data, and the parameter feedback includes motion distance feedback; and the obtaining motion parameter data corresponding to the action and conducting parameter feedback according to the motion parameter data includes obtaining the motion distance data, and conducting the motion distance feedback according to the motion distance data.

In some embodiments, when the action attribute corresponding to the action is the centripetal training, the obtaining the motion distance data, and conducting the motion distance feedback according to the motion distance data includes: obtaining a centripetal motion start point k and a centripetal motion end point k corresponding to the action; obtaining a centripetal motion time k according to the centripetal motion start point k and the centripetal motion end point k; and prompting to speed up the action or slow down the action by comparing the centripetal motion time k with a preset centripetal motion time.

In some embodiments, when the action attribute corresponding to the action is the centrifugal training, the obtaining the motion time data and conducting the motion time feedback according to the motion time data includes: obtaining a centrifugal motion start point k and a centrifugal motion end point k corresponding to the action; obtaining a centrifugal motion time k according to the centrifugal motion start point k and the centrifugal motion end point k; and prompting to speed up the action or slow down the action comparing the centrifugal motion time k with a preset centrifugal motion time.

In some embodiments, when the centripetal motion includes at least two centripetal motion end points and the centrifugal motion includes at least two centrifugal motion start points, and the obtaining the motion time data and conducting the motion time feedback according to the motion time data includes: obtaining an equal length motion time according to a time difference between a last centripetal motion end point corresponding to the centripetal motion and a first centrifugal motion start point corresponding to the centrifugal motion; and comparing the equal length motion time with a preset equal length time, and prompting that a next motion state corresponding to the action is retracted or maintained.

In some embodiments, when the centripetal motion includes at least two centripetal motion distances and the centrifugal motion includes at least two centrifugal motion distances, the obtaining the motion distance data, and conducting the motion distance feedback according to the motion distance data includes: obtaining comprehensive feedback according to motion distance feedback of a last centripetal motion distance and a first centrifugal motion distance.

In some embodiments, the motion parameter data includes motion speed data; and obtaining the motion speed data according to the motion distance data and the motion time data corresponding to the motion distance data, and conducting motion weight feedback according to the motion speed data.

In some embodiments, the conducting the motion weight feedback according to the motion speed data includes: in a group of actions, obtaining an action satisfying preset conditions, the preset conditions including: the comprehensive feedback corresponding to the action is that the action is standard or non-standard, and the action attribute corresponding to the action is the same as a preset action attribute; obtaining an average motion speed of the action satisfying the preset conditions; and comparing the average motion speed with a preset average speed to determine whether to adjust a motion weight corresponding to the action.

In some embodiments, the preset average speed includes a preset average centripetal speed and a preset average centrifugal speed; when the action attribute corresponding to the action is the centripetal training, if the average motion speed corresponding to the centripetal training is greater than a maximum value of the preset average centripetal speed, increasing the motion weight; if the average motion speed corresponding to the centripetal training is less than a minimum value of the preset average centripetal speed, reducing the motion weight; when the action attribute corresponding to the action is the centrifugal training, if the average motion speed corresponding to the centrifugal training is greater than a maximum value of the preset average centrifugal speed, reducing the motion weight; if the average motion speed corresponding to the centrifugal training is less than a minimum value of the preset average centrifugal speed, increasing the motion weight.

### BRIEF DESCRIPTION OF THE DRAWINGS

The present disclosure may be further described in the form of exemplary embodiments, which may be described in detail by the accompanying drawings. These embodiments are not restrictive. In these embodiments, the same number represents the same structure, and the accompanying drawings described here are intended to provide a further understanding of the embodiments of the present disclosure, constitute a part of the application, and do not constitute a limitation of the embodiments of the present disclosure, wherein:

FIG. 1 is an exemplary flowchart of motion parameter feedback according to some embodiments in the present disclosure;

FIG. 2 is a flow diagram of an action feedback method according to some embodiments in the present disclosure;

FIG. 3 is a schematic diagram of an action feedback system according to some embodiments in the present disclosure;

FIG. 4 illustrates a position relationship of a centripetal motion start point, a centripetal motion end point, a centrifugal motion start point, and a centrifugal motion end point in Embodiment 3;

FIG. 5 is a schematic diagram of a current cumulative centripetal motion distance in Embodiment 3;

FIG. 6 is a schematic diagram of a count of each action in Embodiment 3; and

FIG. 7 is a schematic diagram of a centripetal motion time and a centrifugal motion time in Embodiment 3.

### DETAILED DESCRIPTION

The purpose of the present disclosure is to provide multi-dimensional action feedback for all actions covered, and feedback information is more scientific and reasonable for long-term use. Based on this, some embodiments of the present disclosure provide an action feedback method and device.

In order to realize the purpose of the present disclosure, some embodiments of the present disclosure provide an action feedback method, including: judging whether an action is safe; obtaining motion distance data corresponding to the action, and conducting parameter feedback according to the motion distance date; obtaining motion time data of the action, and conducting motion time feedback according to the motion time data; obtain a motion speed according to the motion distance data and motion time data, and conducting motion weight feedback according to the motion speed.

When in use, the action of the user may be obtained through a camera to judge whether the action is safe or not. The judgement of whether the action is safe or not may be achieved by judging, e.g., a position of an arm of the fitness device. This process may refer to a judgment method of whether the action is safe or not in the patent document with the application number of 2021111989366, titled as “action feedback methods, systems, devices, and storage mediums for an intelligent fitness device.”

After judging the safety of the action of the user, the user may perform the action. For a user, when completing a single action, a pull rope with a specific weight may be pulled to a specific position at a specific speed. In this process: the weight may be preset and user is perceivable, and relative motion distance may be obtained by pulling the pull rope to the specific position. This process may be learned in advance and perceived by the user according to a position of the user's body. Speed is a most unobtrusive parameter for user. In theory, if users complete an action in given weight, distance and time as required, the speed is known, so the speed may be used as a feedback parameter of a weight. A most appropriate weight may mean that the user can use this weight to undertake a given count of training, and an average speed of following training after a group of training may decrease slightly. In above parameters, it may be necessary to first determine whether the action is standard, then determine whether the motion speed of the user is appropriate, and finally determine whether the weight needs to be adjusted. The action of the user may be fed back through multiple dimensions, i.e., the weight, speed, and motion distance, making motion training more scientific and improving a use efficiency.

In some embodiments of the present disclosure, the obtaining the motion time data and conducting the motion time feedback according to the motion time data specifically includes: obtaining a centripetal motion start point k and a centripetal motion end point k, where k may be a positive integer; obtaining a centripetal motion distance k according to the centripetal motion start point k and the centripetal motion end point k; obtaining a centrifugal motion start point k and a centrifugal motion end point k corresponding to the action; obtaining a centrifugal motion distance k according to the centrifugal motion start point k and the centrifugal motion end point k.

In some embodiments of the present disclosure, the centripetal motion start point may be a position point where a speed direction is positive and the speed is >2 cm/s, and the centripetal motion end point may be a position point where a speed direction is positive and a speed is <2 cm/s within 100 ms. The centrifugal motion start point is a point where a speed direction is negative and the speed is >2 cm/s; the centrifugal motion end point may be a position point where a speed direction is negative and a speed is less than 1 cm/s within 200 ms. A positive speed direction may be a speed direction towards a user, and a negative speed direction may be a speed direction away from the user. In some embodiments of the present disclosure, a determination of the centrifugal motion end point may be broader than a determination of the centripetal motion end point, because if a centrifugal contraction mode is started, the force may continue to increase in the centrifugal process, during which the user may continue to raise muscle force to fight against it. In an initial stage, the user may have too much resistance due to inadaptation, which may cause the action to stop. Therefore, in order to avoid this situation, some embodiments of the present disclosure may set a broad determination condition of the centrifugal motion end point.

For each action, a centripetal motion distance and a centrifugal motion distance may be calculated to judge whether the action satisfies a threshold requirement, and feedback results may be summarized.

The centripetal motion distance may be a distance from the centripetal motion end point k to the centripetal motion start point k, where k belongs to [1, n], and n is a positive integer. The distance may be greater than 5 cm. Every time a centripetal motion end point k is generated, the centripetal motion distance may be calculated.

The centrifugal motion distance may be a distance from the centrifugal motion end point k to the centrifugal motion start point k, and the distance may be greater than 5 cm. Every time a centrifugal motion end point k is generated, the centrifugal motion distance may be calculated.

In some embodiments of the present disclosure, the motion distances may be calculated by centripetal motion and centrifugal motion respectively, while an existing motion distance may be judged by a preset pull length. Compared with the existing invention, the motion end point may be a position of the pull rope relative to an origin of the pull rope, and the origin of the pull rope may be not absolute, which may be affected by a height of the fitness device hanged on the wall and a height of an arm of the fitness device. For a same action, if a height of the fitness device hanged on the wall and a height of an arm of the fitness device are different, end points that the user should move to may also be different. Thus, a uniform threshold standard may not be used for judgment and feedback. Therefore, the relative motion distance may be adopted. Regardless of the height of the fitness device hanged on the wall and the height of the arm of the fitness device, there may be a standard for the user to complete a whole motion relative to the start point.

The method further includes: obtaining a current cumulative centripetal motion distance according to the centripetal motion distance k; and comparing the current cumulative centripetal motion distance k with a preset centripetal motion distance to judge whether the action is error, standard or non-standard; obtaining a current cumulative centrifugal motion distance according to the centrifugal motion distance k; and comparing the current cumulative centrifugal motion distance with a preset centrifugal motion distance to judge whether the action is error, standard or non-standard.

Specifically, when starting from a centripetal motion start point 1 to a centripetal motion end point 1, a value of the centripetal motion distance 1 may be obtained; when starting from a centripetal motion start point 2 to a centripetal motion end point 2, a value of the centripetal motion distance 2 may be obtained. The current cumulative centripetal motion distance may be a sum of the centripetal motion distance 1 and the centripetal motion distance 2. A reason why the current cumulative centripetal motion distance and current cumulative centrifugal motion distance are used for motion distance feedback may be that if the user stops midway and then pulls to a standard position, if the cumulative motion distances are not used, the motion distance of both parts may not satisfy the requirements, resulting in a prompt that the two actions are nonstandard twice. Prompting the user that the action is nonstandard for the second time is paying for a previous error, which may make the user act “bigger”, cause action deformation. Therefore, it must be determined according to the cumulative motion distances. The influence caused by discontinuous action may be effective in speed determination and weight feedback.

Every time a centripetal motion end point is generated, the current cumulative centripetal motion distance is calculated, and the current cumulative centripetal motion distance is compared with a preset centripetal motion distance. As used herein, the preset centripetal motion distance may include an upper limit of the preset centripetal motion distance and a lower limit of the preset centripetal motion distance. If the current cumulative centripetal motion distance exceeds the upper limit of the preset centripetal motion distance, the action may be reported as error. If the current cumulative centripetal motion distance is lower than the lower limit of the preset centripetal motion distance, the action may be reported as nonstandard, and if the current cumulative centripetal motion distance is within the upper and lower limits of the preset centripetal motion distance, the action may be reported as standard.

Every time a centrifugal motion end point is generated, the current cumulative centrifugal motion distance is calculated, and the current cumulative centrifugal motion distance is compared with a preset centrifugal motion distance. As used herein, the preset centrifugal motion distance may include an upper limit of the preset centrifugal motion distance and a lower limit of the preset centrifugal motion distance. If the current cumulative centrifugal motion distance exceeds the upper limit of the preset centrifugal motion distance, the action may be reported as nonstandard. If the current cumulative centrifugal motion distance is lower than the lower limit of the preset centrifugal motion distance, the action may be reported as error. If the current cumulative centrifugal motion distance is within the upper and lower limits of the preset centrifugal motion distance, the action may be reported as standard.

In some embodiments of the present disclosure, an action may have a plurality of centripetal motion start points, centripetal motion end points, centrifugal motion start points and centrifugal motion end points due to a pause of the action of the user or other factors. Each centripetal motion start point to the centripetal motion end point, and each centrifugal motion start point to the centrifugal motion end point may have a feedback recording.

In some embodiments of the present disclosure, the count may plus 1 from a generation of a centripetal start point to a generation of a first centrifugal motion end point, that is, N times of pulling out to one time of retraction may be determined that an action has been completed. The motion distance and the motion distance feedback may be recorded for each time (counting at an end of centrifugal motion) and a final overall feedback of a single action may be confirmed.

As used herein, if a plurality of centripetal motion distances and centrifugal motion distances are generated in a centripetal or centrifugal motion, comprehensive feedback may be obtained according to the motion distance feedback of a last centripetal motion distance and a first centrifugal motion distance. Specifically, if the feedback in either direction of the centripetal motion distance or centrifugal motion distance is error, the comprehensive feedback may be error; if the feedback in either direction of the centripetal motion distance or centrifugal motion distance is nonstandard, the comprehensive feedback may be nonstandard; if the feedback in either direction of the centripetal motion distance or centrifugal motion distance is standard, the comprehensive feedback may be standard.

After obtaining the motion distance data, it is also necessary to obtain the motion time data to judge the motion speed, and conduct motion time feedback according to the motion time data, specifically as follow steps.

Obtain a centripetal motion time k according to the centripetal motion start point k and the centripetal motion end point k.

Obtain a centrifugal motion time k according to the centrifugal motion start point k and the centrifugal motion end point k.

The centripetal motion time may be a time difference between the centripetal motion start point k and the centripetal motion end point k, k belongs to [1, n], which is calculated when the centripetal motion end point k is generated. The centripetal motion time k may be compared with a preset centripetal motion time. If the centripetal motion time k exceeds an upper limit of the preset centripetal motion time, speeding up the motion may be prompted. If the centripetal motion time k is lower than a lower limit of the preset centripetal motion time, slowing down the motion may be prompted.

The centrifugal motion time may be a time difference between the centrifugal motion start point k and the centrifugal motion end point k, the centrifugal motion time may be calculated when the centrifugal motion end point k is generated. The centrifugal motion time k may be compared with a preset centrifugal time. If the centrifugal motion time k exceeds an upper limit of the preset centrifugal time, speeding up the action may be prompted. If the centrifugal motion time k is lower than the lower limit of the preset centrifugal time, slowing down the action may be prompted.

The equal length motion time may be a time difference between a last centripetal motion end point corresponding to the centripetal motion and a first centrifugal motion start point corresponding to the centrifugal motion. The equal length motion time may be calculated after a distance from the centrifugal motion start point is increased by 5 cm, in which the shaking within 5 cm may be regarded as equal length. According to the time difference between the last centripetal motion end point and the first centrifugal motion start point, the equal length motion time may be obtained, the equal length motion time may be compared with a preset equal length motion time, if the equal length motion time exceeds a preset upper limit of the equal length motion time, the retraction may be prompted, if the equal length motion time is lower than a preset lower limit of the equal length motion time, maintaining the action may be prompted.

After obtaining a plurality of motion time data and feedback on the motion time data, the motion time feedback of each time (counting at the end of the centrifugal motion) may be recorded and the final overall feedback of a single action may be confirmed for subsequent action speed judgment and weight adjustment. If a plurality of centripetal/centrifugal motion time feedback is generated in a centripetal/centrifugal motion, the last centripetal motion and the first centrifugal motion may prevail.

Furthermore, after obtaining the motion time and motion distance, the speed may be obtained, and then an output weight of the pull rope may be adjusted according to the speed, so that the users may be more scientific and reasonable in the process of fitness, and adapt to their own fitness training.

In some embodiments of the present disclosure, it may be determined whether the average speed of a series of actions in the group after the completion of the group of actions that adopt the same settings, that is, actions with the same action attributes, and the comprehensive motion distance feedback is correct or nonstandard conforms to the average speed calculated according to the standard actions. A group of actions may include several actions, the speed may be determined after the group of actions and the weight may be fed back through the speed. This is because: each action already has motion distance feedback and time feedback, if each action has weight feedback, an information density may be too high, which may be of little significance; in addition, under an appropriate training weight, the users may be usually smooth in first few times of pulling the rope, and have a little difficulty in last few times. Therefore, it may be more scientific and reasonable to make a comprehensive judgment after the completion of the group of actions.

Only data with standard or nonstandard comprehensive feedback may be added to the average speed calculation. In the group of actions, actions with standard or nonstandard comprehensive feedback as well as the same action attributes (including training modes) and actions with same settings may be obtained. An average motion speed of the above actions may be obtained. The average motion speed may be compared with a preset average speed to determine whether to adjust the weight.

When the action attribute is the centripetal training, if the average speed is greater than a maximum value of the preset average speed, the motion weight may be increased; if the average speed is less than a minimum value of the preset average speed, the motion weight may be reduced. When the action attribute is the centrifugal training, if the average speed is greater than the maximum value of the preset average speed, the motion weight may be reduced; if the average speed is less than the minimum value of the preset average speed, the motion weight may be increased.

Corresponding to the method in the present disclosure, some embodiments of the present disclosure also provide an action feedback system, including: an action safety module, configured to judge whether the action of the user is safe; an action distance module, configured to obtain the motion distance data of the action of the user; a motion distance feedback module, configured to conduct the motion distance feedback according to the motion distance data to judge the action is error, standard or nonstandard; a motion time module, configured to obtain the motion time data of the action of the user; a motion time feedback module, configured to conduct the motion time feedback according to the motion time data and prompt to speed up or slow down the motion; a motion speed module, configured to obtain motion speed according to the motion distance data and the motion time data; and a motion weight feedback module, configured to judge whether to adjust the weight according to the motion speed and the motion distance feedback module.

Corresponding to the method in the present disclosure, some embodiments of the present disclosure also provide an electronic device, including a memory, a processor, and a computer program stored in the memory and capable of running on the processor. The processor implements the steps of the above action feedback method when executing the computer program.

Corresponding to the method in the present disclosure, some embodiments of the present disclosure also provide a storage medium, the computer-readable storage medium stores a computer program, and the computer program may be executed by a processor to realize the steps of the above action feedback method.

One or more technical solutions provided by some embodiments of the present disclosure at least have the following technical effects or advantages: the action feedback method of the present disclosure may target all actions, and a process of rope pulling training may be classified into the centrifugal motion and centripetal motion, and multi-dimensional feedback may be conducted through the distance, time, speed and weight in the process. The method can provide more scientific and reasonable feedback, further improve the use effect, and facilitate long-term use.

In order to explain the technical scheme of the embodiments of the present disclosure more clearly, the following may briefly introduce the drawings that need to be used in the description of the embodiments. Obviously, the drawings in the following description are only some examples or embodiments of the present disclosure. For those skilled in the art, the present disclosure can also be applied to other similar scenarios according to these drawings without creative work. Unless it is obvious from the language environment or otherwise stated, the same label in the figures represents the same structure or operation.

It should be understood that the “system”, “device”, “unit” and/or “module” used herein is a method for distinguishing different components, elements, parts or assemblies at different levels. However, if other words can achieve the same purpose, they can be replaced by other expressions.

It should be understood by those skilled in the art that in the disclosure of this disclosure, the terms “vertical”, “horizontal”, “top”, “bottom”, “front”, “back”, “left”, “right”, “vertical”, “inside”, “outside”, etc. refer to the orientation or location relationship based on the orientation or location relationship shown in the drawings, which is only for the convenience of describing the present disclosure and simplifying the description, rather than indicating or implying that the device or element referred to must have a specific orientation, be constructed and operate in a specific orientation, the above terms cannot be understood as a limitation of the present disclosure.

As according to the description and the claims, unless the context expressly indicates exceptions, the words “a”, “an”, “the”, “one”, and/or “this” do not specifically refer to the singular, but may also include the plural form; and the plural forms may be intended to include the singular forms as well, unless the context clearly indicates otherwise. Generally speaking, the terms “include” only indicate that the steps and elements that have been clearly identified are included, and these steps and elements do not constitute an exclusive list. Methods or equipment may also include other steps or elements.

A flowchart is used in this disclosure to explain the operation conducted by the system according to the embodiment of the present disclosure. It should be understood that the previous or subsequent operations are not necessarily conducted accurately in order. Instead, the steps may be processed in reverse order or simultaneously. At the same time, you can add other operations to these processes, or remove one or more steps from these processes.

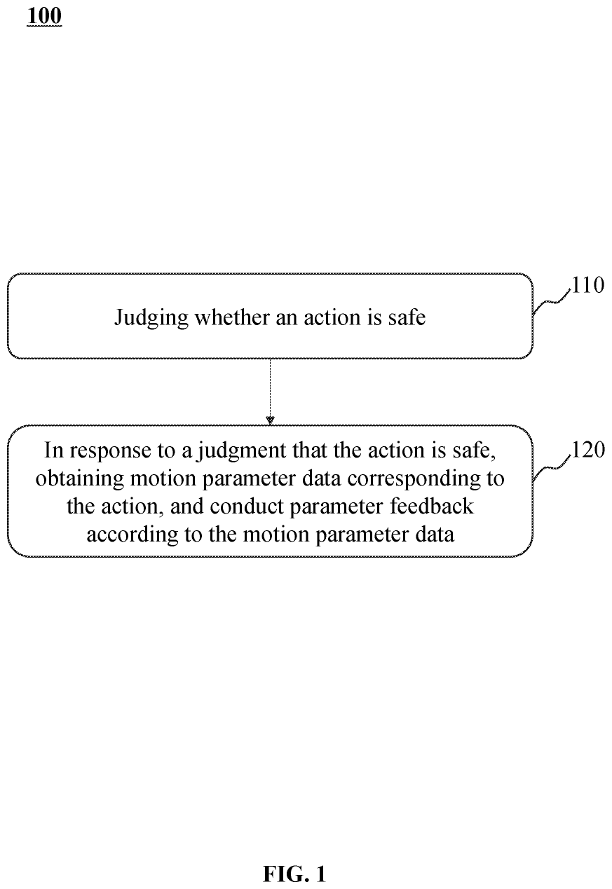

FIG. 1 is an exemplary flowchart of motion parameter feedback according to some embodiments in the present disclosure.

In some embodiments, process 100 may be executed by a processor. As shown in FIG. 1, the process 100 may include following operations.

In step 110, judging whether an action is safe.

In some embodiments, a safety condition for judging whether the action is safe may be preset, and the controller may judge whether the action is safe based on the preset safety condition.

The safety condition may refer to various preset indicators that the motion action does not trigger safety accidents. In some embodiments, the safe condition may be a preset standard for various actions. For example, when the action exceeds a strength range that the user can bear, the action may deviate from a preset action range (for example, deviate from the action axis) or the user's limbs may have a degree of deformation beyond the preset deformation (for example, wrist bending), which may lead to a risk of the safety accidents. In some embodiments, the processor may set different safe conditions according to different users. For example, the safe conditions may be set according to the user's gender, age, height, proficiency, etc.

In some embodiments, when it is judged that the action is unsafe, the controller may generate warning information and prompt the user through a feedback device. The warning information may include voice information, text information, video information, etc. For example, the user may be reminded of potential safe risks of actions by broadcasting equipment, and prompted to correct actions.

In some embodiments, the action may be recognized by motion images. In some embodiments, the controller may acquire motion images. The motion images may be one or more frames of at least one group of actions obtained by an image capturing device (e. g., a camera). The motion images may include at least one group of images of centripetal motion or the at least one group of images of centrifugal motion. In some embodiments, the processor may analyze and process the motion images. For example, the processor may process gray level change, color distortion, etc. of each frame of the motion images, and may also extract motion features of the user in the motion images (for example, types of limb actions, bending, stretching, etc.) for analysis and processing.

In some embodiments, the processor may also compare or match a plurality of frames of motion images with reference images to identify actions in the motion images. By way of example, the processor may compare or match the motion features of the motion images with those of a plurality of reference images in a preset reference image library, determine the reference images matching the motion images, and take actions of the reference images as target actions. As used herein, the reference images may be a plurality of motion images including different preset actions.

In some embodiments, the processor may also recognize and process acquired motion images through a trained action determination model to determine the target actions. As an example, the action determination model may be a convolutional neural network. In the convolutional neural network, input actual motion images and a preset reference motion image may be represented by a picture matrix, and an output of the convolutional neural network may be a similarity of motion action information. A mapping relationship between the actual action images and the preset reference image may be constructed through the convolution neural network to obtain more accurate comparison results.

In step 120, in response to a judgment that the action is safe, obtaining motion parameter data corresponding to the action, and conduct parameter feedback according to the motion parameter data.

The motion parameter data may refer to preset index data of various motions. For example, the motion parameter data may include motion distance data, motion time data, motion speed data, and so on. The motion parameter data may also include other preset parameter data, for example, the motion parameter data may also include a degree of deformation of limbs (for example, arms), an extent of deviation from a preset action axis, etc.

In some embodiments, the processor may also analyze and process a plurality of frames images of a same group of motion images to determine the motion parameters of a same target action. For example, the processor may acquire a plurality of continuous frame motion images from a beginning to an end of images with at least one group of centripetal motion or centrifugal motion (for example, a series of actions to pull out or pull back a rope) for analysis and processing, and determine the motion parameter data of the action.

In some embodiments, in response to the judgment that the action is safe, the processor may obtain the motion parameter data corresponding to the action, and conduct the parameter feedback through the feedback device according to the motion parameter data. For example, you can give prompts or alarms through voice broadcast, microphone, speaker equipment, etc.

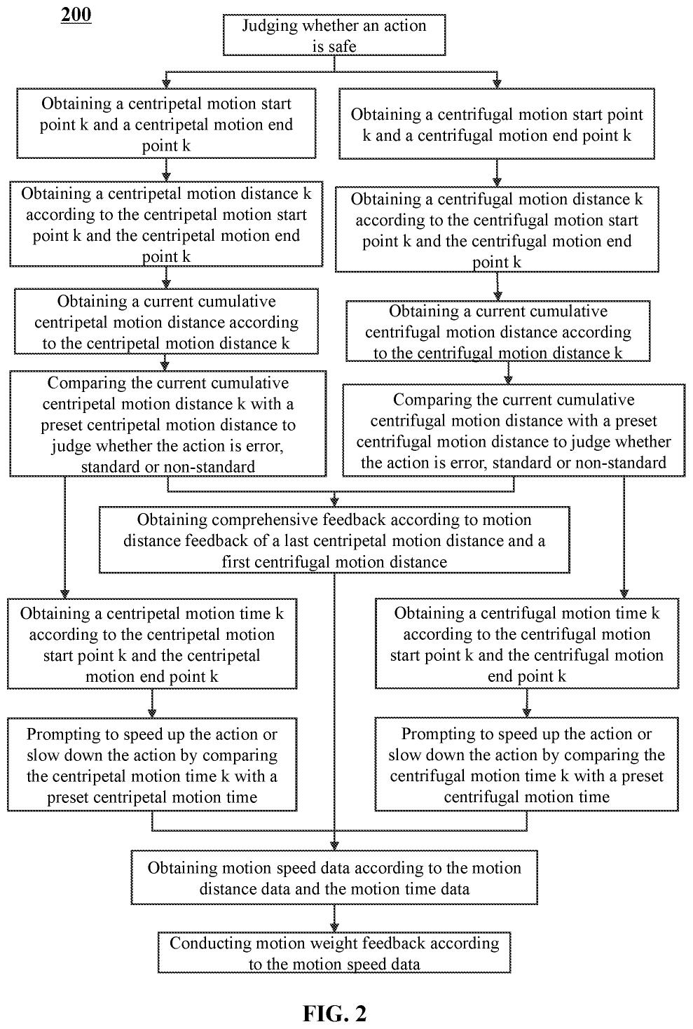

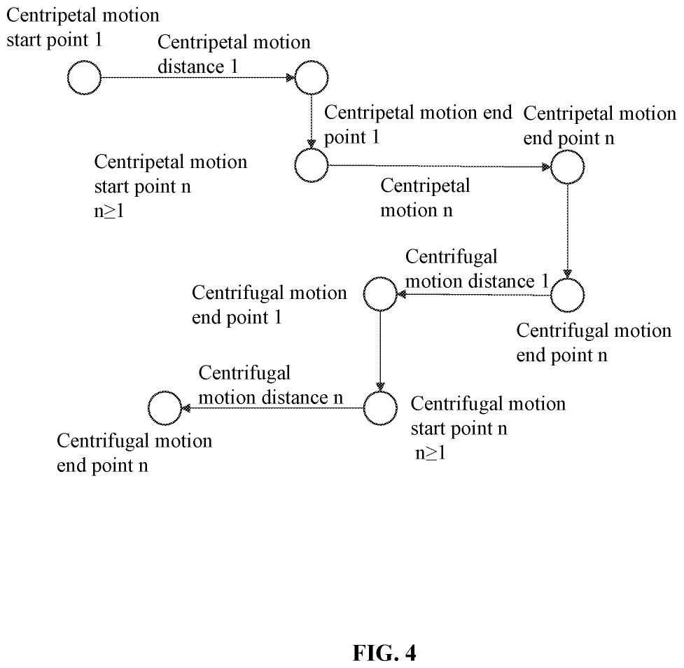

In some embodiments, the parameter feedback may include motion distance feedback, motion time feedback, motion weight feedback, and the like. For more information, please refer to the description of FIG. 2 and FIG. 4.

It should be noted that the above description of process 100 is only for example and description, and does not limit the scope of application of the present disclosure. For those skilled in the art, various modifications and changes can be made to the process under the guidance of the present disclosure. However, these amendments and changes are still within the scope of the present disclosure.

FIG. 2 is a flow diagram of an action feedback method according to some embodiments in the present disclosure. The action feedback method involved in some embodiments of the present disclosure may be described in detail below. It should be noted that following embodiments are only used to explain the present disclosure and do not constitute a limitation of the present disclosure.

### Embodiment 1

Please refer to FIG. 2, FIG. 2 is a flow diagram of an action feedback method. Some embodiments of the present disclosure provide an action feedback method. In this embodiment, the present disclosure is used for a fitness device including a motor, a support arm, and a pull rope coupled to the motor through the arm. When in use, the user may perform fitness training by pulling the pull rope. When pulling the pull rope, the motor may output a force on the pull rope, so that the user can achieve a fitness effect when pulling the rope.

In this embodiment, the process 200 of the action feedback method according to FIG. 2 may be executed by a processor, and the process 200 may include following steps.

Judge whether the action is safe. This step may be used to determine the action of the user, obtain first real-time position data of the arm, and judge whether the action is safe through the first real-time position data.

Obtain the motion distance data of the action, and conduct motion distance feedback according to the motion distance data.

In some embodiments, when the action attribute corresponding to the action is centripetal training, the processor may obtain the motion distance data and conduct motion distance feedback according to the motion distance data. The process 200 may include following steps.

Obtain the centripetal motion start point k and the centripetal motion end point k, where k may be a positive integer.

Obtain a centripetal motion distance k according to the centripetal motion start point k and the centripetal motion end point k.

Obtain a current cumulative centripetal motion distance according to the centripetal motion distance k.

Compare the current cumulative centripetal motion distance. If the current cumulative centripetal motion distance exceeds the upper limit of the preset centripetal motion distance, the action may be reported as error. If the current cumulative centripetal motion distance is lower than the lower limit of the preset centripetal motion distance, the action may be reported as error. If the current cumulative centripetal motion distance is within the upper and lower limits, the action may be reported as standard.

In some embodiments, when the action attribute corresponding to the action is the centrifugal training, the processor may obtain the motion distance data and conduct motion distance feedback according to the motion distance data. The process 200 may also include following steps.

Obtain a centrifugal motion start point k and a centrifugal motion end point k.

Obtain a centrifugal motion distance k according to the centrifugal motion start point k and the centrifugal motion end point k.

Obtain a current cumulative centrifugal motion distance according to the centrifugal motion distance k.

Compare the current cumulative centrifugal motion distance with the preset centrifugal motion distance. If the current cumulative centrifugal motion distance exceeds the upper limit of the preset centrifugal motion distance, the action may be reported as nonstandard. If the current cumulative centrifugal motion distance is lower than the lower limit of the preset centrifugal motion distance, the action may be reported as error. If the current cumulative centrifugal motion distance is within the upper and lower limits, the action may be reported as standard.

In some embodiments, when the action attribute corresponding to the action is the centripetal training or the centrifugal training, the processor may also judge whether the action is standard based on a comparison or matching between the motion image and the reference motion image. For example, the processor may determine whether an action is standard based on difference information between the motion image and the action in the reference motion image (for example, an image of a rope pulling action at the beginning, an image of a holding action at an end, and the like). Exemplary, the reference motion image may be a plurality of preset motion change images in the centripetal training or centrifugal training. The processor may compare a real-time motion image sequence with the reference image. When the difference information is less than a preset difference threshold, the action may be determined as standard. Otherwise, the action may be determined as nonstandard.

In some embodiments, when the action attribute corresponding to the action includes the centripetal training or centrifugal training, the motion parameter data may also include motion time data, the parameter feedback may include the motion time feedback, and the process 200 may also include following steps.

Obtain the motion time data of the action, and conduct the motion time feedback according to the motion time data.

As used herein, when the action attribute corresponding to the action is the centripetal training, the processor may obtain the centripetal motion time k according to the centripetal motion start point k and the centripetal motion end point k.

Compare the centripetal motion time K with the preset centripetal motion time. If the centripetal motion time k exceeds the upper limit of the preset centripetal motion time, prompting to speed up the action. If the centripetal motion time k is lower than the lower limit of the preset centripetal motion time, prompting to slow down the action.

When the action attribute corresponding to the action is the centrifugal training, the processor may obtain the centrifugal motion time k according to the centrifugal motion start point k and the centrifugal motion end point k.

Compare the centrifugal motion time k with the preset centrifugal motion time, and compare the centrifugal motion time k with the preset centrifugal motion time. If the centrifugal motion time k exceeds the upper limit of the preset centrifugal motion time, prompting to speed up the motion.

Obtain the motion speed according to the motion distance data and motion time data, and conduct the motion weight feedback according to the motion speed.

As used herein, the comprehensive feedback may be obtained according to the motion distance feedback of the last centripetal motion distance and the first centrifugal motion distance.

In a group of actions, actions satisfying preset conditions may be obtained. The actions of preset conditions may include actions whose corresponding comprehensive feedback is standard or nonstandard and whose action attributes are the same.

Obtain an average motion speed of the above actions.

Compare the average motion speed with the preset average speed to determine whether to adjust the weight.

In some embodiments, when the action attribute corresponding to the action is the centripetal training, if the average motion speed is greater than the maximum value of the preset average speed, the motion weight may be increased; if the average motion speed is less than the minimum value of the preset average speed, the motion weight may be reduced.

In some embodiments, when the action attribute corresponding to the action is the centrifugal training, if the average motion speed is greater than the maximum value of the preset average speed, the motion weight may be reduced; if the average motion speed is less than the minimum value of the preset average speed, the motion weight may be increased.

### Embodiment 2

On a basis of Embodiment 1, the action feedback method of the present disclosure is introduced in combination with below specific examples:

In step 1, adjusting the position of the support arm.

In step 2, judging whether the action is safe.

In step 3, obtaining the motion distance data of the action, and conduct motion distance feedback according to the motion distance data.

In step 3.1, obtaining a centrifugal motion start point k and a centripetal motion end point k. As shown in FIG. 4, the centripetal motion start point may be a position point where a speed direction is a positive direction, that is, the user pulls out the rope, and a speed of the starting point is >2 cm/s, and the centripetal motion end point may be a position point where a speed direction is positive and a speed of the centripetal motion end point is less than 2 cm/s within 100 ms; the centrifugal motion start point may be a position point where a speed direction is negative, that is, a direction of the user to recover the rope, and the speed is >2 cm/s; the centrifugal motion end point may be a position point where a speed direction is negative and a speed is less than 1 cm/s for 200 ms. Where k belongs to [1, n], and n is a positive integer greater than or equal to 1.

In step 3.2, obtaining a centripetal motion distance, that is, a distance from the centripetal motion end point k to the centripetal motion start point k. Where, k belongs to [1, n], and n is a positive integer, and the distance is greater than 5 cm. Calculate the centripetal motion distance every time a centripetal motion end point k is generated.

Obtaining a centrifugal motion distance, that is, a distance from the centrifugal motion end point k to the centrifugal motion start point k, and the distance is greater than 5 cm. Calculating the centrifugal motion distance every time a centrifugal motion end point k is generated.

In step 3.3, obtaining motion distance feedback.

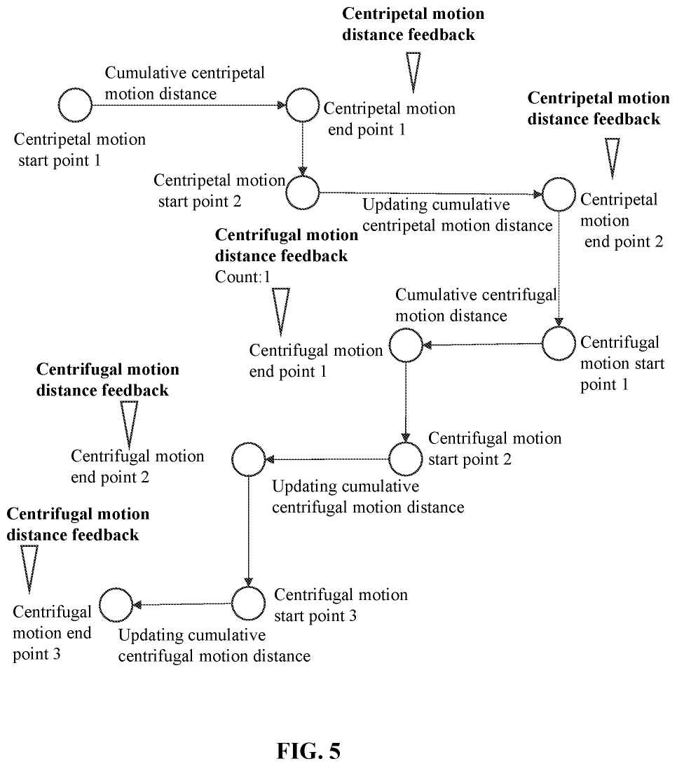

In step 3.31, as shown in FIG. 5, every time a centripetal motion end point is generated, calculating the current cumulative centripetal motion distance, and comparing the current cumulative centripetal motion distance with a preset centripetal motion distance. As used herein, the preset centripetal motion distance may include the upper limit of the preset centripetal motion distance and the lower limit of the preset centripetal motion distance. If the current cumulative centripetal motion distance exceeds the upper limit of the preset centripetal motion distance, the action may be reported as error. If the current cumulative centripetal motion distance is lower than the lower limit of the preset centripetal motion distance, the action may be reported as nonstandard, and if the current cumulative centripetal motion distance is within the upper and lower limits, the action may be reported as standard.

In step 3.32, every time a centrifugal motion end point is generated, calculating the current cumulative centrifugal motion distance, and comparing the current cumulative centrifugal motion distance and a preset centrifugal motion distance. If the current cumulative centrifugal motion distance exceeds an upper limit of the preset centrifugal motion distance, the action may be reported as nonstandard. If the current cumulative centrifugal motion distance is lower than a lower limit of the preset centrifugal motion distance, the action may be reported as error. If the current cumulative centrifugal motion distance is within the upper and lower limits, the action may be reported as standard.

In step 3.4, obtaining comprehensive feedback based on motion distance feedback.

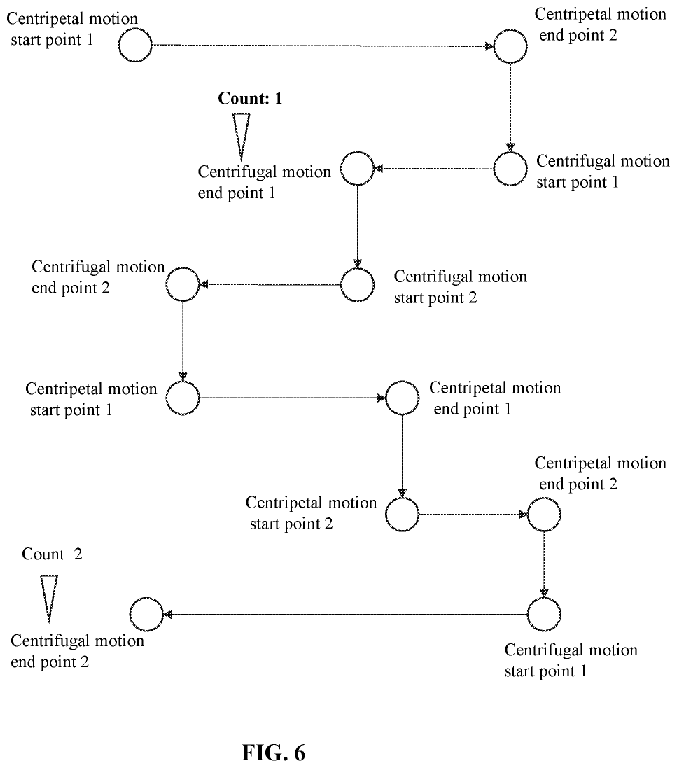

In step 3.41, as according to FIG. 6, the count may plus 1 from a time when a centripetal motion start point is generated to a time when a first centrifugal motion end point is generated, that is, after N times of pulling out to one time of retraction, it is determined that an action has been completed. Record a motion distance and motion distance feedback for each time (counting at an end of centrifugal motion) and confirm a final overall feedback of a single action; Moreover, if the plurality of centripetal motion distances and centrifugal motion distances are generated in a centripetal or centrifugal motion, comprehensive feedback may be obtained according to the motion distance feedback of a last centripetal motion distance and a first centrifugal motion distance. In this embodiment, the comprehensive feedback is according to Table 1:

TABLE 1

| Scene | 1 | 2 | 3 | 4 | 5 | 6 | 7 | 8 | 9 |
|---|---|---|---|---|---|---|---|---|---|
| Last feedback | Standard | Nonstandard | Error | Standard | Nonstandard | Error | Standard | Nonstandard | error |
| of the centripetal | | | | | | | | | |
| motion | | | | | | | | | |
| First feedback | Standard | Standard | Standard | Error | Error | Error | Nonstandard | Nonstandard | nonstandard |
| of the centrifugal | | | | | | | | | |
| motion | | | | | | | | | |
| Comprehensive | Standard | Nonstandard | Error | Error | Error | Error | Nonstandard | Nonstandard | error |
| feedback | | | | | | | | | |

In step 4, obtaining motion time data of the action, and conducting motion time feedback according to the motion time data.

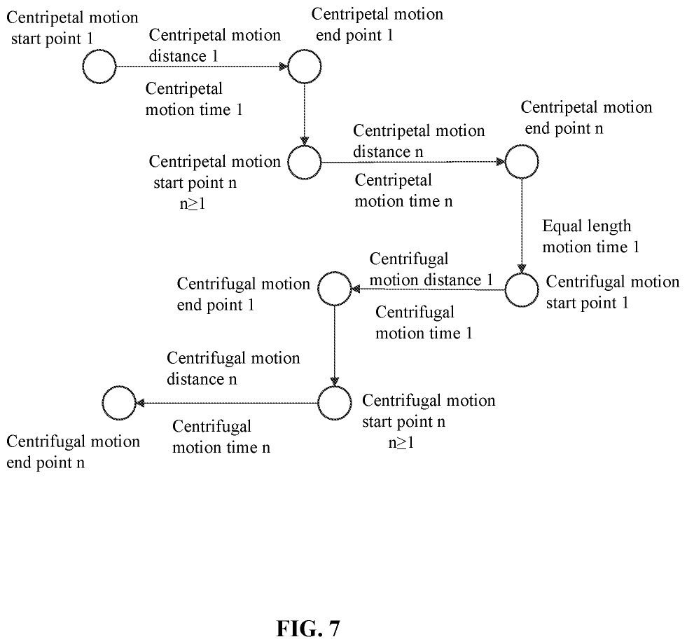

In step 4.1, as according to FIG. 7, obtaining the centripetal motion time k according to the centripetal motion start point k and the centripetal motion end point k. The centripetal motion time may be a time difference between the centripetal motion start point k and the centripetal motion end point k, k belongs to [1, n], which is calculated when the centripetal motion end point k is generated.

In step 4.11, comparing the centripetal motion time k with the preset centripetal motion time. If the centripetal motion time k exceeds the upper limit of the preset centripetal motion time, prompting to speed up the action; if the centripetal motion time k is lower than the lower limit of the preset centripetal motion time, prompting to slow down the action.

In step 4.2, as according to FIG. 7, the centrifugal motion time k may be obtained according to the centrifugal motion start point k and the centrifugal motion end point k. The centrifugal motion time may be a time difference between the centrifugal motion start point k and the centrifugal motion end point k, the centrifugal motion time may be calculated when the centrifugal motion end point k is generated.

In step 4.21, comparing the centrifugal motion time k with a preset centrifugal time, if the centrifugal motion time k exceeds an upper limit of the preset centrifugal time, prompting to speed up the action, if the centrifugal motion time k is lower than the upper limit of the preset centrifugal time, prompting to slow down the action.

In step 4.3, as according to FIG. 7, obtaining an equal length motion time according to a time difference between a last centripetal motion end point and a first centrifugal motion start point. The equal length motion time may be the time difference from the last centripetal motion end point to the first centrifugal motion start point. The equal length motion time may be calculated after a distance from the centrifugal motion start point is increased by 5 cm, in which the shaking within 5 cm may be regarded as equal length.

In step 4.31, comparing the equal length motion time with a preset equal length motion time, if the equal length motion time exceeds a preset upper limit of the equal length motion time, prompting to retract the action, and if the equal length motion time is lower than a preset lower limit of the equal length motion time, prompting to maintain the action.

In this embodiment, the preset time is shown in Table 2:

TABLE 2

| Preset | | Preset equal | | Preset | |
|---|---|---|---|---|---|
| centrifugal time | | length motion time | | centrifugal time | |
| Lower | Upper | Lower | Upper | Lower | Upper |
| limit | limit | limit | limit | limit | limit |
| 0.8 s | 1.2 s | 1.6 s | 2.4 s | 3.6 s | 4.8 s |

In step 4.4, recording the motion time feedback of each time (counting at an end of the centrifugal motion) and confirming the final overall feedback of a single action.

In step 5, obtaining a motion speed according to the motion distance data and the motion time data, and conducting motion weight feedback according to the motion speed.

In step 5.1, in a group of actions, obtaining comprehensive feedback as standard or nonstandard actions with the same action attribute.

In step 5.2, obtaining the action attribute of the action in step 5.1, and judging whether there is a centrifugal contraction mode, if there is the centrifugal contraction mode, only determining the speed in a centrifugal phase; if there is no centrifugal contraction mode, judging that there is the centripetal training, and only determining the speed in the centripetal phase.

In step 5.3, obtaining an average centripetal motion distance or an average centrifugal motion distance, and corresponding average centripetal motion time or average centrifugal motion time according to the step 5.2.

In step 5.4, obtaining an average centripetal motion speed or an average centrifugal motion speed according to the step 5.3.

V_{average centripetal motion}=average centripetal motion distance in the group of actions/average centripetal motion time in the group of actions.

V_{average centrifugal motion}=average centrifugal motion distance in the group of actions/average centrifugal motion time in the group of actions.

In step 5.5, comparing the average motion speed with the preset average speed.

When the action attribute is the centripetal training, if the average motion speed is greater than a maximum value of the preset average speed, then increasing a motion weight; if the average motion speed is less than a minimum value of the preset average speed, then reducing the motion weight.

When the action attribute is the centrifugal training, if the average motion speed is greater than the maximum value of the preset average speed, then reducing the motion weight; if the average motion speed is less than the minimum value of the preset average speed, then increasing the motion weight.

Specifically, in this embodiment, including following operations.

m_set: a centripetal basic weight set by the user.

Δm_set: a weight change of a training mode set by the user.

m_adjust: a centripetal basic weight after an adjustment of the action weight.

Δm_adjust: a change weight after an adjustment of the action weight.

fb % (feedback): a proportion to be adjusted.

When the action attribute corresponding to the action is the centripetal training, following operations may be included.

(1) When an average speed V_{average centrifugal motion} of an action pulled by the user is greater than a maximum value Vconmax of the average centripetal speed in the group, the weight may need to be increased, the processor may determine the increased weight value based on following formulas (1.1), (1.2) and (1.3):

$$fb \% = \qquad ( 1.1 )$$

$$min ( 50 \% , rounddown ( \frac{Vaverage centrifugal motion - Vcon max}{6} \times 5 \% ) )$$

$$m\_adjust = max ( 2 kg , round ( m\_set \times ( 1 + fb \% ) ) ) \qquad ( 1.2 )$$

$$Δm\_adjust = max ( 0 kg , round ( Δm\_set \times ( 1 + fb \% ) ) ) \qquad ( 1.3 )$$

(2) When the average speed V_{average centrifugal motion} of the action pulled by the user is less than a minimum value Vconnmin of the average centripetal speed in the group, the weight may need to be reduced, the processor may determine the reduced weight value based on following formulas (2.1), (2.2) and (2.3):

$$fb \% = \qquad ( 2.1 )$$

$$min ( 50 \% , rounddown ( \frac{V con min - Vaverage centrifugal motion}{6} \times 5 \% ) )$$

$$m\_adjust = max ( 2 kg , round ( m\_set \times ( 1 - fb \% ) ) ) \qquad ( 2.2 )$$

$$Δm\_adjust = max ( 0 kg , round ( Δm\_set \times ( 1 - fb \% ) ) ) \qquad ( 2.3 )$$

When the action attribute corresponding to the action is the centrifugal training, following operations may be included.

(1) When an average speed V_{average centrifugal motion} of an action retracted by the user is greater than a maximum value Veccmax of the average centrifugal speed in the group, the weight may need to be reduced, the processor may determine the reduced weight value based on following formulas (3.1), (3.2) and (3.3):

$$fb \% = \qquad ( 3.1 )$$

$$min ( 50 \% , rounddow ( \frac{Vaverage centrifugal motion - Vecc max}{6} \times 5 \% ) )$$

$$m\_adjust = max ( 2 kg , round ( m\_set \times ( 1 - fb \% ) ) ) \qquad ( 3.2 )$$

$$Δm\_adjust = max ( 0 kg , round ( Δm\_set \times ( 1 - fb \% ) ) ) \qquad ( 3.3 )$$

(2) When the average speed V_{average centrifugal motion} of an action retracted by the user is smaller than a minimum value Veccm in of the average centrifugal speed in the group, the weight may need be increased, the processor may determine the increased weight value based on following formulas (4.1), (4.2) and (4.3):

$$fb \% = \qquad ( 4.1 )$$

$$min ( 50 \% , rounddown ( \frac{Vecc min - Vaverage centrifugal motion}{6} \times 5 \% ) )$$

$$m\_adjust = max ( 2 kg , round ( m\_set \times ( 1 + fb \% ) ) ) \qquad ( 4.2 )$$

$$Δm\_adjust = max ( 0 kg , round ( Δm\_set \times ( 1 + fb \% ) ) ) \qquad ( 4.3 )$$

For example, a first group of actions may correspond to the centripetal training, the first group of actions may include 5 actions, and the specific information is shown in Table 3:

TABLE 3

| | Count of groups | | | | |
|---|---|---|---|---|---|
| | The first group | | | | |
| | Count | | | | |
| | 1 | 2 | 3 | 4 | 5 |
| Training mode | Exhausted | Chain | Chain | Chain | Chain |
| Basic weight + | a + c | a + b | a + c | a + c | a + c |
| change weight | | | | | |
| Centripetal | 90 cm | 80 cm | 98 cm | 90 cm | 86 cm |
| motion distance | | | | | |
| Centripetal | 1.6 | 2.2 | 2 | 1.6 | 1.8 |
| motion time | | | | | |
| Comprehensive | Standard | Nonstandard | Error | Standard | Nonstandard |
| feedback | | | | | |

According to the Table 3, the actions satisfying the requirements of the step 5.1 are the fourth and fifth actions. The average centripetal motion distance in the group is obtained according to the Table 3. The average centripetal motion distance in the group=Avg (90,86)=88 cm.

The average centripetal motion time of the fourth and fifth motions in the group is equal to: Avg (1.6,1.8)=1.7 s.

The average centripetal speed V_{average centripetal motion} in the group of the fourth and fifth actions is equal to 88/1.7=51.8 cm/s.

The V_{average centripetal motion} is compared with the preset average speed. In this embodiment, the preset average speed of the group of actions may be 74.2 cm/s-111.2 cm/s, so 51.8<74.2 (weight reduction required).

### Embodiment 3

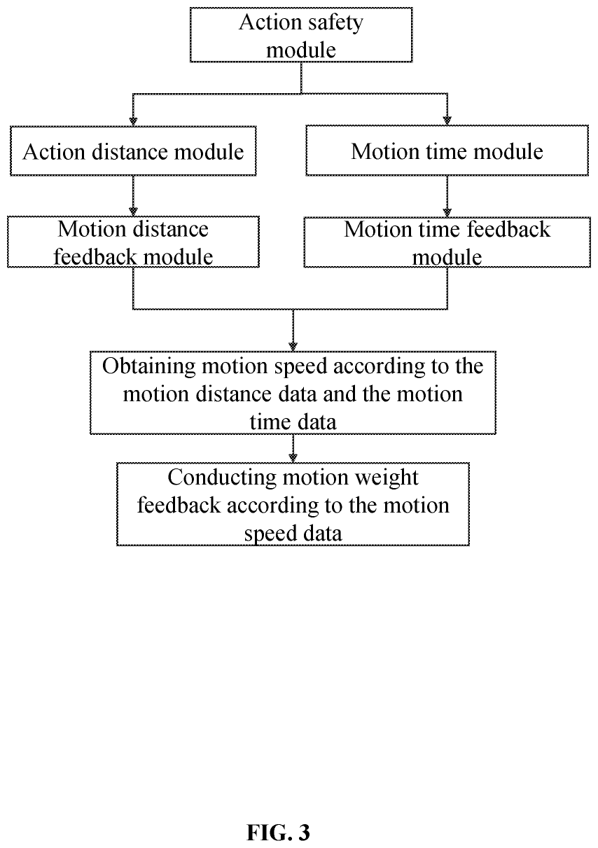

Please refer to FIG. 3, FIG. 3 is a schematic diagram of an action feedback system according to some embodiments in the present disclosure. Embodiment 3 of the present disclosure provides an action feedback system. On a basis of the above examples, the system may include following modules:
an action safety module, configured to judge whether the action of the user is safe;
an action distance module, configured to obtain the motion distance data of the action of the user;
a motion distance feedback module, configured to conduct the motion distance feedback according to the motion distance data to judge whether the action is error, standard, or nonstandard;
a motion time module, configured to obtain the motion time data of the action of the user;
a motion time feedback module, configured to conduct the motion time feedback according to the motion time data and prompt to speed up or slow down the motion;
a motion speed module, configured to obtain motion speed according to the motion distance data and the motion time data; and
a motion weight feedback module, configured to judge whether to adjust the weight according to the motion speed and the motion distance feedback module.

### Embodiment 4

Embodiment 4 of the present disclosure provides an electronic device, including a memory, a processor, and a computer program stored in the memory and capable of running on the processor. The processor may implement steps of the action feedback method when executing the computer program.

As used herein, the processor may be a central processor, or other general-purpose processors, digital signal processors, special integrated circuits, off the shelf programmable gate arrays or other programmable logic devices, discrete gates or transistor logic devices, discrete hardware components, etc. A general-purpose processor may be a microprocessor or any conventional processor or the like.

The memory may be configured to store the computer program and/or module, and the processor may realize various functions of an action feedback device in the present disclosure by running or executing data stored in the memory. The memory may mainly include a storage program area and a storage data area, wherein the storage program area may store an operating system, an application program required for at least one function (such as a sound playing function, an image playing function, etc.), etc. In addition, the memory may include high-speed random access memory, and may also include non-volatile memory, such as hard disk, memory, plug-in hard disk, smart memory card, security digital card, flash memory card, at least one disk storage device, flash memory device, or other volatile solid-state storage devices.

### Embodiment 5

Embodiment 5 of the present disclosure provides a computer-readable storage medium, storing a computer program. When the computer program is executed by a processor, steps of the action feedback method may be implemented.

The computer storage medium of the embodiment of the present disclosure may adopt any combination of one or more computer-readable media. The computer-readable medium may be a computer-readable signal medium or a computer-readable storage medium. The computer-readable storage medium may include, but not limited to, an electrical, magnetic, optical, electromagnetic, infrared, or semiconductor system, device, or device, or any combination thereof. More specific examples of computer-readable storage media (non-exhaustive examples) may include: electrical connection with one or more wires, portable computer disk, hard disk, random access memory (RAM), read-only memory (ROM), erasable programmable read-only memory ((EPROM) or flash memory), optical fiber, portable compact disk read-only memory (CD-ROM), optical storage device Magnetic storage device, or any suitable combination of the above. In the present disclosure, the computer-readable storage medium may be any tangible medium containing or storing a program, which may be used by or in combination with an instruction execution system, device, or instrument.

The basic concepts have been described above. Obviously, for those skilled in the art, the above detailed disclosure is only an example and does not constitute a limitation of the present disclosure. Although it is not explicitly stated here, those skilled in the art may make various modifications, improvements, and amendments to the present disclosure. Such modifications, improvements and amendments are suggested in the present disclosure, so such modifications, improvements and amendments still belong to the spirit and scope of the exemplary embodiments of the present disclosure.

Meanwhile, the present disclosure uses specific words to describe the embodiments of the present disclosure. For example, “one embodiment”, and/or “some embodiments” mean a certain feature or structure related to at least one embodiment of the present disclosure. Therefore, it should be emphasized and noted that “one embodiment” or “an alternative embodiment” mentioned twice or more in different positions in the present disclosure does not necessarily refer to the same embodiment. In addition, certain features or structures in one or more embodiments of the present disclosure may be appropriately combined.

In addition, unless explicitly stated in the claims, the sequence of processing elements and sequences, the use of numbers and letters, or the use of other names described in the present disclosure are not used to define the sequence of processes and methods in the present disclosure. Although the above disclosure has discussed some currently considered useful embodiments of the present disclosure through various examples, it should be understood that such details are only for the purpose of explanation, and the additional claims are not limited to the disclosed embodiments. On the contrary, the claims are intended to cover all amendments and equivalent combinations that conform to the essence and scope of the embodiments of the present disclosure. For example, although the system components described above can be implemented by hardware devices, they can also be implemented only by software solutions, such as installing the described system on an existing server or mobile device.

Similarly, it should be noted that, in order to simplify the description disclosed in the present disclosure and thus help the understanding of one or more embodiments of the present disclosure, the foregoing description of the embodiments of the present disclosure sometimes incorporates a variety of features into one embodiment, the drawings or the description thereof. However, this disclosure method does not mean that the object of the present disclosure requires more features than those mentioned in the claims. In fact, the features of the embodiments are less than all the features of the single embodiments disclosed above.

In some embodiments, numbers describing the number of components and attributes are used. It should be understood that such numbers used in the description of embodiments are modified by the modifier “about”, “approximate” or “generally” in some examples. Unless otherwise stated, “approximately” or “generally” indicate that a ±20% change in the FIG. is allowed. Accordingly, in some embodiments, the numerical parameters used in the description and claims are approximate values, and the approximate values can be changed according to the characteristics required by individual embodiments. In some embodiments, the numerical parameter should consider the specified significant digits and adopt the method of general digit reservation. Although the numerical fields and parameters used to confirm the range breadth in some embodiments of the present disclosure are approximate values, in specific embodiments, the setting of such values is as accurate as possible within the feasible range.

For each patent, patent application, patent application disclosure and other materials cited in the present disclosure, such as articles, books, specifications, publications, documents, etc., the entire contents are hereby incorporated into the present disclosure for reference. Except for the application history documents that are inconsistent with or conflict with the contents of the present disclosure, and the documents that limit the widest range of claims in the present disclosure (currently or later appended to the present disclosure). It should be noted that in case of any inconsistency or conflict between the description, definitions, and/or use of terms in the supplementary materials of the present disclosure and the contents described in the present disclosure, the description, definitions, and/or use of terms in the present disclosure shall prevail.

Finally, it should be understood that the embodiments described in the present disclosure are only used to illustrate the principles of the embodiments of the present disclosure. Other deformations may also fall within the scope of the present disclosure. Therefore, as an example rather than a limitation, the alternative configuration of the embodiments of the present disclosure can be regarded as consistent with the teachings of the present disclosure. Accordingly, the embodiments of the present disclosure are not limited to those explicitly introduced and described in the present disclosure.

What is claimed is:

1. An action feedback method, wherein the method includes:
judging whether an action is safe; and
obtaining motion parameter data corresponding to the action in response to a judgment that the action is safe, and conducting parameter feedback according to the motion parameter data.

2. The action feedback method of claim 1, wherein an action attribute corresponding to the action includes centripetal training or centrifugal training, the motion parameter data includes motion distance data, and the parameter feedback includes motion distance feedback; and
the obtaining motion parameter data corresponding to the action and conducting parameter feedback according to the motion parameter data includes obtaining the motion distance data, and conducting the motion distance feedback according to the motion distance data.

3. The action feedback method of claim 2, wherein
when the action attribute corresponding to the action is the centripetal training, the obtaining the motion distance data and conducting the motion distance feedback according to the motion distance data includes:
obtaining a centripetal motion start point k and a centripetal motion end point k corresponding to the action;
obtaining a centripetal motion distance k according to the centripetal motion start point k and the centripetal motion end point k;
obtaining a current cumulative centripetal motion distance according to the centripetal motion distance k; and
comparing the current cumulative centripetal motion distance k with a preset centripetal motion distance to judge whether the action is error, standard or non-standard; and
when the action attribute corresponding to the action is the centrifugal training, the obtaining the motion distance data and conducting the motion distance feedback according to the motion distance data includes:
obtaining a centrifugal motion start point k and a centrifugal motion end point k corresponding to the action;
obtaining a centrifugal motion distance k according to the centrifugal motion start point k and the centrifugal motion end point k;
obtaining a current cumulative centrifugal motion distance according to the centrifugal motion distance k; and
comparing the current cumulative centrifugal motion distance with a preset centrifugal motion distance to judge whether the action is error, standard or non-standard.

4. The action feedback method of claim 1, wherein the action attribute corresponding to the action includes centripetal training or centrifugal training, the motion parameter data includes motion time data, and the parameter feedback includes motion time feedback; and
the obtaining motion parameter data corresponding to the action and conducting parameter feedback according to the motion parameter data includes obtaining the motion time data and conducting the motion time feedback according to the motion time data.

5. The action feedback method of claim 4, wherein when the action attribute corresponding to the action is the centripetal training, the obtaining the motion time data and conducting the motion time feedback according to the motion time data includes:
obtaining a centripetal motion start point k and a centripetal motion end point k corresponding to the action;
obtaining a centripetal motion time k according to the centripetal motion start point k and the centripetal motion end point k; and
prompting to speed up the action or slow down the action by comparing the centripetal motion time k with a preset centripetal motion time; and
when the action attribute corresponding to the action is the centrifugal training, the obtaining the motion time data and conducting the motion time feedback according to the motion time data includes:
obtaining a centrifugal motion start point k and a centrifugal motion end point k corresponding to the action;
obtaining a centrifugal motion time k according to the centrifugal motion start point k and the centrifugal motion end point k; and
prompting to speed up the action or slow down the action by comparing the centrifugal motion time k with a preset centrifugal motion time.

6. The action feedback method of claim 4, wherein when the centripetal motion includes at least two centripetal motion end points and the centrifugal motion includes at least two centrifugal motion start points, and the obtaining the motion time data and conducting the motion time feedback according to the motion time data includes:
obtaining an equal length motion time according to a time difference between a last centripetal motion end point corresponding to the centripetal motion and a first centrifugal motion start point corresponding to the centrifugal motion; and
comparing the equal length motion time with a preset equal length time, and prompting that a next motion state corresponding to the action is retracted or maintained.

7. The action feedback method of claim 2, wherein when the centripetal motion includes at least two centripetal motion distances and the centrifugal motion includes at least two centrifugal motion distances, the obtaining the motion distance data, and conducting the motion distance feedback according to the motion distance data includes:
obtaining comprehensive feedback according to motion distance feedback of a last centripetal motion distance and a first centrifugal motion distance.

8. The action feedback method of claim 7, wherein the motion parameter data includes motion speed data; and
obtaining the motion speed data according to the motion distance data and the motion time data corresponding to the motion distance data, and conducting motion weight feedback according to the motion speed data.

9. The action feedback method of claim 8, wherein the conducting the motion weight feedback according to the motion speed data includes:
in a group of actions, obtaining an action satisfying preset conditions, the preset conditions including: the comprehensive feedback corresponding to the action is that the action is standard or non-standard, and the action attribute corresponding to the action is the same as a preset action attribute;
obtaining an average motion speed of the action satisfying the preset conditions; and
comparing the average motion speed with a preset average speed to determine whether to adjust a motion weight corresponding to the action.

10. The action feedback method of claim 9, wherein the preset average speed includes a preset average centripetal speed and a preset average centrifugal speed;
when the action attribute corresponding to the action is the centripetal training, if the average motion speed corresponding to the centripetal training is greater than a maximum value of the preset average centripetal speed, increasing the motion weight; if the average motion speed corresponding to the centripetal training is less than a minimum value of the preset average centripetal speed, reducing the motion weight;
when the action attribute corresponding to the action is the centrifugal training, if the average motion speed corresponding to the centrifugal training is greater than a maximum value of the preset average centrifugal speed, reducing the motion weight; if the average motion speed corresponding to the centrifugal training is less than a minimum value of the preset average centrifugal speed, increasing the motion weight.

11. An action feedback device, including at least one storage medium and at least one processor:
at least one storage device including a group of instructions; and
at least one processor communicating with the at least one storage device, wherein when executing the group of instructions, the at least one processor is configured to enable the system to perform operations, including:
judging whether an action is safe; and
obtaining motion parameter data corresponding to the action in response to a judgment that the action is safe, and conducting parameter feedback according to the motion parameter data.

12. The action feedback device of claim 11, wherein an action attribute corresponding to the action includes centripetal training or centrifugal training, the motion parameter data includes motion distance data, and the parameter feedback includes motion distance feedback; and
the obtaining motion parameter data corresponding to the action and conducting parameter feedback according to the motion parameter data includes obtaining the motion distance data, and conducting the motion distance feedback according to the motion distance data.

13. The action feedback device of claim 11, wherein when the action attribute corresponding to the action is the centripetal training, the obtaining the motion distance data, and conducting the motion distance feedback according to the motion distance data includes:
obtaining a centripetal motion start point k and a centripetal motion end point k corresponding to the action;
obtaining a centripetal motion distance k according to the centripetal motion start point k and the centripetal motion end point k;
obtaining a current cumulative centripetal motion distance according to the centripetal motion distance k; and
comparing the current cumulative centripetal motion distance with a preset centripetal motion distance to judge whether the action is error, standard or non-standard; and
when the action attribute corresponding to the action is the centrifugal training, the obtaining the motion distance data and conducting the motion distance feedback according to the motion distance data includes:
obtaining a centrifugal motion start point k and a centrifugal motion end point k corresponding to the action;
obtaining a centrifugal motion distance k according to the centrifugal motion start point k and the centrifugal motion end point k;
obtaining a current cumulative centrifugal motion distance according to the centrifugal motion distance k; and
comparing the current cumulative centrifugal motion distance with a preset centrifugal motion distance to judge whether the action is error, standard or non-standard.

14. The action feedback device of claim 11, wherein the action attribute corresponding to the action includes centripetal training or centrifugal training, the motion parameter data includes motion time data, and the parameter feedback includes motion time feedback; and
the obtaining motion parameter data corresponding to the action and conducting parameter feedback according to the motion parameter data includes obtaining the motion time data and conducting the motion time feedback according to the motion time data.

15. The action feedback device of claim 14, wherein when the action attribute corresponding to the action is the centripetal training, the obtaining the motion time data and conducting the motion time feedback according to the motion time data includes:
obtaining a centripetal motion start point k and a centripetal motion end point k corresponding to the action;
obtaining a centripetal motion time k according to the centripetal motion start point k and the centripetal motion end point k; and
prompting to speed up the action or slow down the action by comparing the centripetal motion time k with a preset centripetal motion time; and
when the action attribute corresponding to the action is the centrifugal training, the obtaining the motion time data and conducting the motion time feedback according to the motion time data includes:
obtaining a centrifugal motion start point k and a centrifugal motion end point k corresponding to the action;
obtaining a centrifugal motion time k according to the centrifugal motion start point k and the centrifugal motion end point k; and
prompting to speed up the action or slow down the action comparing the centrifugal motion time k with a preset centrifugal motion time.

16. The action feedback device of claim 14, wherein when the centripetal motion includes at least two centripetal motion end points and the centrifugal motion includes at least two centrifugal motion start points, and the obtaining the motion time data and conducting the motion time feedback according to the motion time data includes:
obtaining an equal length motion time according to a time difference between a last centripetal motion end point corresponding to the centripetal motion and a first centrifugal motion start point corresponding to the centrifugal motion; and
comparing the equal length motion time with a preset equal length time, and prompting that a next motion state corresponding to the action is retracted or maintained.

17. The action feedback device of claim 12, wherein when the centripetal motion includes at least two centripetal motion distances and the centrifugal motion includes at least two centrifugal motion distances, the obtaining the motion distance data, and conducting the motion distance feedback according to the motion distance data includes:
obtaining comprehensive feedback according to motion distance feedback of a last centripetal motion distance and a first centrifugal motion distance.

18. The action feedback device of claim 17, wherein the motion parameter data includes motion speed data; and
obtaining the motion speed data according to the motion distance data and the motion time data corresponding to the motion distance data, and conducting motion weight feedback according to the motion speed data.

19. The action feedback device of claim 18, wherein the conducting the motion weight feedback according to the motion speed data includes:
in a group of actions, obtaining an action satisfying preset conditions, the preset conditions including: the comprehensive feedback corresponding to the action is that the action is standard or non-standard, and the action attribute corresponding to the action is the same as a preset action attribute;
obtaining an average motion speed of the action satisfying the preset conditions; and
comparing the average motion speed with a preset average speed to determine whether to adjust a motion weight corresponding to the action.

20. The action feedback device of claim 19, wherein the preset average speed includes a preset average centripetal speed and a preset average centrifugal speed;
when the action attribute corresponding to the action is the centripetal training, if the average motion speed corresponding to the centripetal training is greater than a maximum value of the preset average centripetal speed, increasing the motion weight; if the average motion speed corresponding to the centripetal training is less than a minimum value of the preset average centripetal speed, reducing the motion weight;
when the action attribute corresponding to the action is the centrifugal training, if the average motion speed corresponding to the centrifugal training is greater than a maximum value of the preset average centrifugal speed, reducing the motion weight; if the average motion speed corresponding to the centrifugal training is less than a minimum value of the preset average centrifugal speed, increasing the motion weight.
